(19) United States
(12) Patent Application Publication
(10) Pub. No.: US 2007/0037268 A1
(43) Pub. Date: Feb. 15, 2007

(54) HYDROGEN PRODUCING BIOREACTOR WITH SAND FOR THE MAINTENANCE OF A HIGH BIOMASS BACTERIA

(76) Inventors: Mitchell S. Felder, Hermitage, PA (US); Harry R. Diz, Erie, PA (US); Justin Felder, Hermitage, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/463,141

(22) Filed: Aug. 8, 2006

Related U.S. Application Data
(60) Provisional application No. 60/706,681, filed on Aug. 9, 2005.

(51) Int. Cl. C12P 3/00; C12M 3/00

(52) U.S. Cl. 435/168; 435/289.1

(57) ABSTRACT

This invention discloses an apparatus for the concentrated growth of hydrogen generating microorganisms. The apparatus includes a receptacle with an interior cavity that contains a material in which the hydrogen generating microorganisms are located as well as one or more substrates that are used to facilitate the growth of hydrogen generating microorganisms.

### CROSS REFERENCE TO RELATED APPLICATIONS

This invention claims priority under 119(e) from U.S. Provisional Application No. 60/706,681, which was filed on Aug. 9, 2005 and is incorporated by reference herein.

### BACKGROUND OF THE INVENTION

1. Field of the Invention

The present invention relates generally to an apparatus for concentrated growth of hydrogen generating microorganism cultures. More particularly, this invention relates to an apparatus for the concentrated growth of hydrogen generating nonparaffinophilic microorganisms.

2. Description of the Related Art

The production of hydrogen gas is an increasingly common and important procedure in the world today. Production of hydrogen in the United States alone currently amounts to about 3 billion cubic feet (ft³) per year, with output likely to increase. Uses for the produced hydrogen are varied, ranging from utilization in welding processes to the production of hydrochloric acid. An increasingly important use of hydrogen, however, is in the production of alternative fuels for machinery, such as motor vehicles. Successful use of hydrogen as an alternative fuel can provide substantial benefits to the world at large. This is possible not only because hydrogen is produced without dependence on the location of specific oils or other ground resources, but because burning hydrogen is atmospherically clean due to the fact that essentially no carbon dioxide or greenhouse gasses are produced when hydrogen is burned. Thus, production of hydrogen as a fuel source can have great impact on the world at large.

Traditional apparatuses of hydrogen production, however, utilize significant amounts of fossil fuels in order to produce hydrogen. For instance, an electrolyzer, which generally uses electricity to decompose water into hydrogen and oxygen, requires a significant amount of fossil fuel to generate the electricity needed to power the decomposition process. Similarly, a steam reformer, which is another apparatus used to produce hydrogen, also requires a large amount of fossil fuel during the hydrogen producing process. As could be readily understood, the environmental benefits of producing hydrogen using these apparatuses and processes are partially offset because these apparatuses and processes utilize significant amounts of pollution-causing fuels, such as fossil fuels, as an energy source during hydrogen production. Accordingly, there is a need for a hydrogen producing apparatus that significantly reduces the consumption of fossil fuel during the hydrogen production process.

### SUMMARY OF THE INVENTION

This need and others are met by various embodiments of this invention which provide an apparatus for the concentrated growth of hydrogen producing microorganisms.

In accordance with one embodiment of the invention, an apparatus for the concentrated growth of a hydrogen generating microorganism includes a receptacle that contains a material with the hydrogen generating microorganism. A substrate that contains a baiting material, which baits the hydrogen generating microorganism to the substrate and allows for the concentrated growth of the microorganism on the substrate, is also contained within the receptacle.

In accordance with another embodiment of the invention, an apparatus for the concentrated growth of a hydrogen generating microorganism includes a receptacle that contains sand. The sand contains the hydrogen generating microorganism. A substrate that contains a baiting material, which baits the hydrogen generating microorganism to the substrate and allows for the concentrated growth of the microorganism on the substrate, is also contained within the receptacle.

In accordance with yet another embodiment of the invention, an apparatus for the concentrated growth of a hydrogen generating nonparaffinophilic microorganism includes a receptacle that contains a material with the hydrogen generating nonparaffinophilic microorganism. A substrate that has a substantially hollow interior contains a baiting material, which baits the hydrogen generating nonparaffinophilic microorganism to the substrate and allows for the concentrated growth of the nonparaffinophilic microorganism on the substrate, is also contained within the receptacle. The baiting material is a mixture of a carbon compound, agar, and water.

One aspect to this invention is to reduce the amount of fossil fuel needed to produce hydrogen.

Another aspect to this invention is to provide an apparatus for the concentrated growth of hydrogen producing microorganisms.

Yet another aspect to this invention is to provide a hydrogen producing apparatus the yields a large of amount of hydrogen while minimizing the monetary costs associated with operating and maintaining such an apparatus.

### BRIEF DESCRIPTION OF THE DRAWINGS

A full understanding of the disclosed and claimed concept can be gained from the following Description when read in conjunction with the accompanying drawings in which:

FIG. 1 is a schematic of the hydrogen producing apparatus in accordance with one embodiment of the invention;

FIG. 2 is depicts one embodiment of the substrate that can be used in the apparatus of FIG. 1; and

FIG. 3 is depicts the substrate of FIG. 2 in the receptacle.

### DESCRIPTION OF THE PREFERRED EMBODIMENTS

As used herein, the term “hydrogen producing microorganisms” shall refer broadly to microorganisms that produce hydrogen gas as a result of their metabolic processes.

When referring to any numerical range of values, such ranges are understood to include each and every number and/or fraction between the stated range minimum and maximum.

Directional phrases used herein, such as, for example, upper, lower, left, right, vertical, horizontal, top, bottom, above, beneath, clockwise, counterclockwise and derivatives thereof, relate to the orientation of the elements shown in the drawings and are not limiting upon the claims unless expressly recited therein.

As stated above, traditional apparatuses and processes used to produce hydrogen consume a large amount of fossil fuel. The apparatus and method that is disclosed in this invention, however, significantly reduces the amount of fossil fuel that is consumed during the hydrogen producing process by facilitating the growth of hydrogen producing (generating) microorganisms such as, but not limited to, nonparaffinophilic bacteria that belong to the genus bacillus, clostridium, and klebsiella as well as other nonparaffinophilic microorganisms.

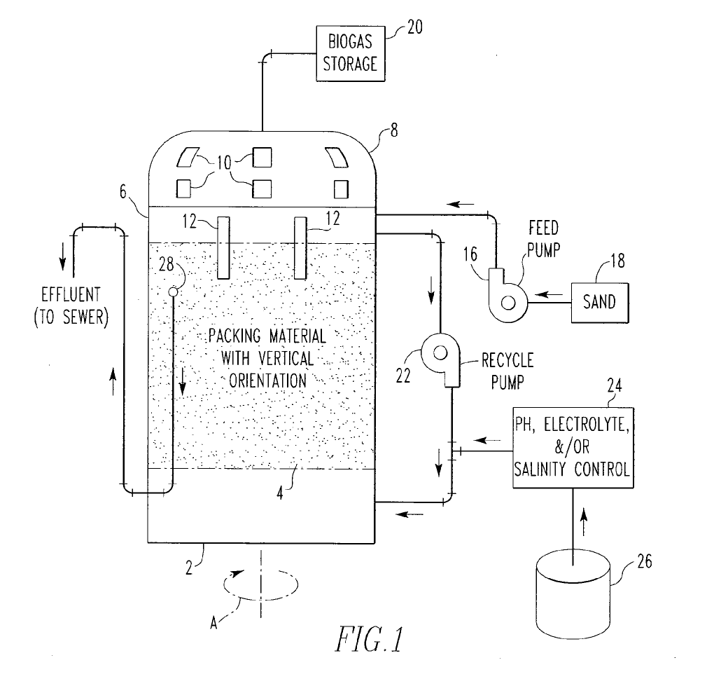

Referring to FIG. 1, a receptacle 2 (hereinafter, also referred to as the bioreactor) that is suitable for containing a material 4 within an interior cavity 6 of the receptacle 2 is provided. The size and shape of the receptacle 2 is not meant to be limiting. It should be noted, however, that as the size (e.g., diameter, height) of the receptacle 2 is increased the concentration of hydrogen producing microorganisms that are grown within the receptacle 2 can also be increased thereby generating a larger amount of hydrogen gas in the receptacle 2 which can be collected.

In one embodiment of the invention, the interior cavity 6 of the receptacle 2 can be accessed through a removable lid 8. In yet another embodiment of the invention, the interior cavity 6 of the receptacle 2 is accessed through one or more access ports 10 that are disposed on the lid 8 and/or the receptacle 2 thereby allowing for the insertion and removal of one or more substrates 12 from the receptacle 2 without having to remove the lid 8 from the receptacle 2. The receptacle 2 can also have means for periodically mixing the contents of the receptacle 2. For instance, the receptacle 2 can be mounted on an apparatus that allows the receptacle 2 to rotate about 180° or 360° in the direction of arrow A. Alternatively, the receptacle 2 can be equipped with a stirrer that is positioned within the interior cavity 6 of the receptacle 2. The stirrer can be moved using mechanical or magnetic means thereby stirring the contents of the receptacle 2. Additionally, the interior cavity 6 of the receptacle 2 may also be lined with a coating of alginate which provides a substrate on which concentrated growth of the hydrogen producing microorganisms can occur.

As will be discussed in greater detail below, the receptacle 2 may be equipped with one or more heating elements that are used to heat the material 4 that is contained within the receptacle 2. In order to regulate the temperature of the material 4, the receptacle 2 may also be equipped with means, such as a thermostat, to control the temperature of the material 4 to facilitate the growth of the hydrogen producing microorganisms in the receptacle 2 and/or to prevent the proliferation of other microorganisms that produce unwanted metabolic by-products such as methane gas. For example, hydrogen producing bacteria can typically withstand temperatures up to about 100° C. for a period ranging from about 3 hours to about 5 hours. In contrast, methane producing bacteria, such as methanogens, cannot withstand temperatures near 100° C. for more than a few minutes. Accordingly, in one embodiment of the invention, the material 4 is heated to a temperature ranging from about 90° C. to about 100° C. for a period of time ranging from about 20 minutes to about 300 minutes or, more preferably, a period of time ranging from about 20 minutes to about 30 minutes. By eliminating the bacteria that produced unwanted by-products from the material 4, the hydrogen that is ultimately produced by the hydrogen producing bacteria will not require additional purification steps in order to separate the hydrogen gas from other gases that would have been produced by the other bacteria. Because additional purification steps are not required, the cost associated with producing hydrogen using the apparatus that is disclosed in this invention is significantly less than other apparatuses that are traditionally used to produce hydrogen.

The material 4 that is introduced into the interior cavity 6 of the receptacle 2 is a material 4 that contains hydrogen producing microorganisms. The material 4, for example, can be sand or a mixture of sand and water since sand has been shown to harbor significant amounts of bacteria. For example, typical beach sand has been shown to have 5 to 10 times the amount of bacteria than water. Moreover, unlike an open water environment, bacteria can survive in sand for months at a time. Because sand naturally contains high concentrations of bacteria, the likelihood of sand containing some amount of hydrogen producing bacteria that can be grown and cultivated within the receptacle 2 is quite high. However, in order to ensure that sufficient concentrations of hydrogen producing microorganisms will be produced in the receptacle 2, the material 4 can be inoculated with additional hydrogen producing microorganisms prior to or after the introduction of the material 4 into the receptacle 2.

Continuing with FIG. 1, one or more passageways 14 such as, without limitation, pipes or tubes are used to connect the receptacle 2 to other components of the apparatus. For example, the material 4 is introduced into the interior cavity 6 of the receptacle 2 by a feed pump 16 that is connected to both the receptacle 2 as well as to the source of the material 18. A storage tank 20, which collects the gas that is produced by the hydrogen producing microorganisms, is connected to the receptacle 2 as well. A recycle pump 22, which is attached to the receptacle 2, is used to recycle additional organic material that is introduced into the receptacle 2. The additional organic material, which is used as a source of nutrients by the hydrogen producing microorganisms, is made from a material that can be metabolized by the hydrogen producing microorganisms. Continuing with FIG. 1, means 24, such as a pump, for controlling the pH, electrolyte levels, and the salinity of the material 4 is connected to the recycle pump 22. A source 26 of another material, such as a Bicarbonate solution, may also be connected to the means 24. As is known in the art, a Bicarbonate solution is used to adjust the pH of a material. However, any solution that is utilized for pH adjustment may be used in lieu of the Bicarbonate solution. Referring to FIG. 1, the receptacle 2 can also include an effluent outlet 28 for the removal of excess liquid and waste from the receptacle 2 to a remote location.

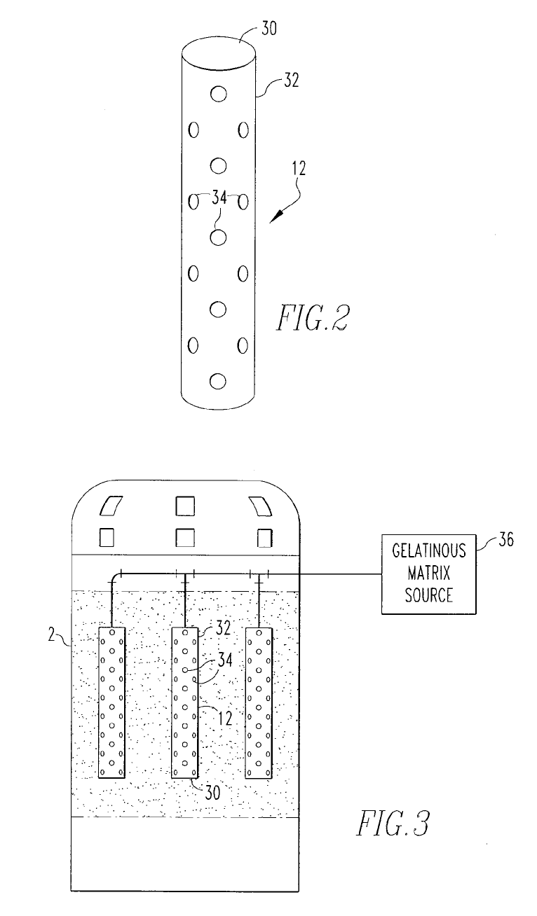

Referring to FIGS. 2 and 3, concentrated growth of the hydrogen producing microorganisms is achieved not only by adjusting the material's environmental conditions (e.g. temperature, pH, salinity, number of electrolytes) to optimize the hydrogen producing microorganisms' growth, but it is also achieved by introducing one or more substrates 12 into the interior cavity of the receptacle 2. In one embodiment of the invention, the substrate 12 has a hollow or partially hollow interior 30 (hereinafter, referred to as the interior of the substrate) that is connected to the exterior surface 32 of the substrate 12 by a plurality of channels 34 that extend from the interior 30 of the substrate to the exterior surface 32. However, a substrate 12 having a solid interior or a substrate having a solid interior with holes or passages disposed on the exterior surface 32 of the substrate 12 can also be used in this invention. The substrate 12 is typically made from a material, such as plastic, which can withstand heat up to about 110° C. so that the substrate 12 can withstand any processes that are designed to eliminate bacteria that produce unwanted by-products (see preceding paragraphs). The substrate 12 can be virtually of any shape including, but not limited to, pipe, rod, bead, slat, tube, screen, honeycomb, sphere, or a shape with latticework. As stated above, the substrates 12 are typically inserted through access ports 10 that are disposed on the receptacle 2 and/or lid 8. It should be noted, however, that the substrate 12 can also be affixed to the interior cavity 6 of the receptacle 2 either in direct contact with or substantially adjacent to the material 4 that is contained within the receptacle 2.

The substrate 12 is coated with a carbon-based baiting material that is used to cultivate the hydrogen producing microorganisms on the substrate 12 by allowing the microorganisms to obtain nutrients directly from the substrate 12 and to form a biofilm on the substrate 12. Preferably, the carbon-based baiting material is a gelatinous matrix having at least one carbon compound. The carbon compound can be selected from the group comprising, but not limited to, glucose, fructose, glycerol, mannitol, asparagines, casein, adonitol, l-arabinose, cellobiose, dextrose, dulcitol, d-galactose, inositol, inulin, lactose, levulose, maltose, d-mannitol, d-mannose, melibiose, raffinose, rhamnose, sucrose, salicin, d-sorbintol, trihalose and d-xylose or any combination thereof. In general, the gelatinous matrix can be prepared by placing about 2 grams (g) of agar and 3 grams (g) of a carbon compound into 100 milliliters (mL) of distilled water. The ratio of agar, carbon compound, and water can be used to scale the amount of the baiting material to the desired levels. It should be noted, however, that the agar, which is a gelatinous mix, can be replaced with other gelatinous mixes that are commonly known in the art.

Once the agar, carbon compound, and distilled water have been mixed, the mixture is boiled for about 20 minutes to about 30 minutes and steamed sterilized for about 20 minutes to about 30 minutes to form a molten gelatinous matrix. While the gelatinous matrix is in molten form, the gelatinous matrix is coated onto the substrate 12. The molten gelatinous matrix can either be applied directly to the surface of the substrate 12 or it can be applied to an adhesive layer that is disposed between the coating and the substrate 12 using methods that are well known in the art. If an adhesive is used, the adhesive is of a type that is commonly known in the art for containing carbon based compounds. For example, the adhesive can be in a form a gel bead that is made from organic glue having affixed thereto, either ionically or by affinity, a carbon compound.

When the level of gelatinous matrix on the substrate 12 becomes too low, additional gelatinous matrix can be coated onto the substrate 12 to ensure that the there is sufficient carbon-based baiting material on the substrate 12 to sustain the growth of the hydrogen producing microorganisms on the substrate 12. This can be achieved by either removing the substrate 12 from the receptacle 2 and coating the substrate 12 with additional gelatinous matrix while it is outside of the receptacle 2 or, as can be seen from FIG. 3, the substrate 12 can be connected to a source 36 of the gelatinous matrix thereby allowing for the replenishment of the gelatinous matrix on the substrate 12 without having to remove the substrate 12 from the receptacle 2. For example, a pump, which is attached to the substrate 12 as well as to the source 36 of the gelatinous matrix, can be used to introduce additional gelatinous matrix onto the substrate 12.

If the concentration of hydrogen producing microorganisms needs to be increased in order to increase hydrogen output, then additional substrates 12 may be introduced into the receptacle 2. The additional substrates 12 can either have a surface area that is equal to the surface area of the substrates 12 that are currently in the receptacle 2 or the additional substrates 12 can have a surface area that is greater than the surface area of the substrates 12 that are in the receptacle 2. If a substrate 12 having a greater surface area is introduced into the receptacle 2, one would appreciate that a higher concentration of hydrogen producing microorganisms can be cultivated on that particular substrate 12 when compared to a substrate 12 with a smaller surface area.

While specific embodiments of the invention have been described in detail above, it will be appreciated by those skilled in the art that various modifications and alternatives to those details could be developed in light of the overall teachings of the disclosure. Accordingly, the particular arrangements disclosed are meant to be illustrative only and not limiting as to the scope of the disclosed and claimed concept which is to be given the full breadth of the claims appended and any and all equivalents thereof.

What is claimed is:

1. An apparatus for the concentrated growth of a hydrogen generating microorganism, said apparatus comprising:
a receptacle;
a material contained within said receptacle, said material contains said hydrogen generating microorganism; and
a substrate contained within said receptacle, said substrate containing a baiting material that baits said hydrogen generating microorganism to said substrate thereby allowing for the concentrated growth of said hydrogen generating microorganism on said substrate.

2. The apparatus according to claim 1, wherein said material is sand.

3. The apparatus according to claim 1, wherein said material is a mixture of sand and water.

4. The apparatus according to claim 1, wherein said hydrogen generating microorganism is a nonparaffinophilic bacteria.

5. The apparatus according to claim 1, wherein said substrate is in direct contact with said material.

6. The apparatus according to claim 1, further comprising a storage tank gaseously connected to said receptacle whereby said storage tank collects the hydrogen that is produced by said hydrogen generating microorganism.

7. The apparatus according to claim 1, wherein said substrate has a substantially hollow interior that is connected to an exterior surface of said substrate by one or more channels.

8. The apparatus according to claim 7, wherein said substrate is connected to a source of said baiting material thereby allowing for the continuous replenishment of said baiting material on said substrate.

9. The apparatus according to claim 1, further comprising a feed pump that is connected to said receptacle and to a source of said material, said feed pump removes said material from said source and introduces said material into said receptacle.

10. The apparatus according to claim 1, further comprising means for adjusting the pH, temperature, salinity, and oxygen levels of said material.

11. The apparatus according to claim 1, further comprising means for stirring said material in said receptacle.

12. The apparatus according to claim 1, wherein said baiting material comprises a carbon compound, agar, and water.

13. The apparatus according to claim 12, wherein said carbon compound is selected from glucose, fructose, glycerol, mannitol, asparagines, casein, adonitol, l-arabinose, cellobiose, dextrose, dulcitol, d-galactose, inositol, inulin, lactose, levulose, maltose, d-mannitol, d-mannose, melibiose, raffinose, rhamnose, sucrose, salicin, d-sorbintol, trihalose and d-xylose or any combination thereof.

14. An apparatus for the concentrated growth of a hydrogen generating microorganism, said apparatus comprising:
a receptacle;
a material contained within said receptacle, said material being sand that contains said hydrogen generating microorganisms; and
a substrate contained within said receptacle, said substrate containing a baiting material that baits said hydrogen generating microorganism to said substrate thereby allowing for the concentrated growth of said hydrogen generating microorganism on said substrate.

15. The apparatus according to claim 14, wherein said bating material comprises a carbon compound, agar, and water.

16. The apparatus according to claim 14, further comprising means for collecting hydrogen that is produced by said hydrogen generating microorganism.

17. The apparatus according to claim 14, further comprising means for adjusting the temperature of said sand.

18. An apparatus for the concentrated growth of a hydrogen generating nonparaffinophilic microorganism, said apparatus comprising:
a receptacle;
a material contained within said receptacle, said material being sand that contains said hydrogen generating nonparaffinophilic microorganism;
a substrate contained within said receptacle, said substrate having a substantially hollow interior; and
a baiting material disposed on said substrate, said baiting material baits said hydrogen generating nonparaffinophilic microorganism to said substrate thereby allowing for the concentrated growth of said hydrogen generating nonparaffinophilic microorganism on said substrate, said baiting material being a mixture of a carbon compound, agar, and water.

19. The apparatus according to claim 18, further comprising means for adjusting the pH, temperature, salinity, and oxygen levels of said material.

20. The apparatus according to claim 19, wherein said carbon compound is selected from glucose, fructose, glycerol, mannitol, asparagines, casein, adonitol, l-arabinose, cellobiose, dextrose, dulcitol, d-galactose, inositol, inulin, lactose, levulose, maltose, d-mannitol, d-mannose, melibiose, raffinose, rhamnose, sucrose, salicin, d-sorbintol, trihalose and d-xylose or any combination thereof.
